(19) United States
(12) Patent Application Publication
(10) Pub. No.: US 2015/0011797 A1
(43) Pub. Date: Jan. 8, 2015

(54) OXIDATION OF CYCLOALKANES IN THE PRESENCE OF A SUPPORTED BIMETALLIC GOLD-PALLADIUM CATALYST

(71) Applicant: INVISTA NORTH AMERICA S.A R.L., Wilmington, DE (US)

(72) Inventors: Keith Whiston, Darlington (GB); Xi Liu, Cardiff (GB); Graham Hutchings, Cardiff (GB)

(73) Assignee: INVISTA NORTH AMERICA S.A R.L., Wilmington, DE (US)

(21) Appl. No.: 14/375,512

(22) PCT Filed: Feb. 1, 2013
(86) PCT No.: PCT/IB2013/050866

§ 371 Date: Jul. 30, 2014

(30) Foreign Application Priority Data
Feb. 3, 2012 (GB) 1201866.9

(51) Int. Cl. C07C 45/32; C07C 29/52; C07C 29/50

(52) U.S. Cl. CPC C07C 45/32; C07C 29/50; C07C 29/52
USPC 568/359; 568/836; 568/837; 568/360

(57) ABSTRACT

The present invention relates to a process for the oxidation of cycloalkanes utilising a supported gold and palladium catalyst and the use of the supported gold and palladium catalyst for the oxidation of cycloalkanes. Also described is a process for the preparation of the supported catalyst.

This invention relates to a process for the oxidation of cycloalkanes utilising a supported gold and palladium catalyst and the use of the supported gold and palladium catalyst for the oxidation of cycloalkanes. Also described is a process for the preparation of the catalyst.

The oxidation of cyclohexane, particularly under mild reaction conditions, is of key importance to industry because the products, in particular cyclohexanol and cyclohexanone, are precursors for the production of adipic acid which is a key intermediate in the production of polyamides, polyurethanes, polyesters and plasticizers. Typically, adipic acid is prepared by oxidising a mixture of cyclohexanol and cyclohexanone (KA oil) with nitric acid. KA oil is the primary product of cyclohexane oxidation. Cyclohexanone is also used as a starting material for caprolactam.

Currently, commercial cyclohexane oxidation is conducted by using homogeneous cobalt and manganese salts, at temperatures above 423K. The process provides cyclohexanol and cyclohexanone as products with selectivity of 85%. Significant amounts of cyclohexyl hydrogen peroxide (CHHP) and trace amounts of carboxylic acid by-products are also produced. However, the high selectivity for cyclohexanol and cyclohexanone products is only obtained at low conversion levels, typically less than 10%, and most typically less than 5% conversion. While manipulation of the reaction conditions can provide greater conversion, this is normally accompanied by deterioration in the selectivity to cyclohexanol and cyclohexanone.

It has been found that catalytic performance above 5% conversion can be achieved in the oxidation of cyclohexane using hydrogen peroxide or tert-butyl hydrogen peroxide (TBHP). However, it is commercially attractive to employ oxygen or air in any industrial process due the cheapness and feasibility in large scale operations. For similar reasons, and from an environmental perspective, it is also desirable to employ a process which does not use solvent.

Environmental issues are also relevant when considering the suitability of catalysts for large-scale production. Higher turn-over-frequency can be obtained by using low concentration of homogeneous catalysts. However, recycling of homogeneous catalysts and disposal of waste liquid are problematic and have significant impacts on the environment. Most conventional heterogeneous catalysts are typically inactive under mild reaction conditions due to relatively low mobility of lattice oxygen.

The catalyst and processes of the invention address the above problems associated with current commercial catalysts and processes for the oxidation of cycloalkanes. Thus, it is an object of this invention to provide a process for cycloalkane (particularly cyclohexane) oxidation which yields cycloalkanol and cycloalkanone (particularly cyclohexanol and cyclohexanone) products with high selectivity (at least 85%, and preferably at least 90%) and at conversion levels above 5% (particularly above 10%). which would be a significant development over current industrial processes. It is a further object of the invention to provide a heterogeneous catalyst which can be recycled and which may be used in a process for oxidising cycloalkanes, which is suitable for industrial scale-up and which yields cycloalkanol and/or cycloalkanone with favourable selectivity and conversion. It is a particular object of the invention to provide a heterogeneous catalyst which can selectively decompose CHHP in a single unit operation, in order to increase conversion and maintain or improve the reaction selectivity to cyclohexanol and cyclohexanone in the oxidation of cyclohexane. It is a further particular object of the invention to provide a more efficient and economical process for the production of adipic acid from the oxidation of cyclohexane. A key element of the overall cost for the current industrial production of adipic acid is steam usage and the alternative catalyst system provided by the present invention significantly reduces this element.

In a first aspect, the invention provides a process for oxidising cycloalkane(s) comprising contacting one or more cycloalkane(s) with an oxidant in the presence of a supported catalyst, wherein the supported catalyst comprises a catalyst comprising gold-palladium particles and a support selected from carbides, nitrides and oxides, wherein the oxides are selected from magnesium, aluminium and zinc oxides.

There is also described a process for the preparation of a supported catalyst comprising the steps of:
(a) preparing an aqueous solution of gold and palladium salts; and
(b) adding a support thereto, wherein the support is selected from carbides, nitrides and oxides, wherein the oxides are selected from magnesium, aluminium and zinc oxides,
   and wherein said process further comprises the step of reducing the gold and palladium ions.

The reduction step typically comprises the addition of a suitable reducing agent, either before or after the addition of the support, and in a preferred embodiment the reducing agent is added before the addition of the support. Alternatively, the reduction step may be effected by calcination. The reduction step reduces the gold and palladium ions to the gold-palladium alloy of the catalyst of the present invention.

In a second aspect, the invention provides an oxidation process for preparing cycloalkanol(s) and/or cycloalkanone(s) comprising contacting one or more cycloalkane(s) with an oxidant in the presence of a supported catalyst, wherein the supported catalyst comprises a catalyst comprising gold-palladium particles and a support selected from carbides, nitrides and oxides, wherein the oxides are selected from magnesium, aluminium and zinc oxides.

In a third aspect, the invention provides the use of a supported catalyst, wherein the supported catalyst comprises a catalyst comprising gold-palladium particles and a support selected from carbides, nitrides and oxides, wherein the oxides are selected from magnesium, aluminium and zinc oxides, as a catalyst for the oxidation of cycloalkanes.

The Catalyst

The gold-palladium particles of the catalyst are a gold-palladium (AuPd) alloy, and preferably in the form of nanoparticles. The mean longest diameter of a nanoparticle is preferably no more than about 200 nm, more preferably no more than about 50 nm, more preferably no more than about 30 nm, and more preferably no more about 20 nm, and most preferably in the range of from about 5 nm to about 15 nm. The mean size and size distribution of the nanoparticles is suitably measured by STEM (Scanning Transmission Electron Microscopy), where visual inspection of a typical section of the supported catalyst sample is used to count particles of different sizes within a given cross sectional area.

The composition of the gold-palladium alloy influences the catalytic activity and the inventors have found that optimum conversion and selectivity in the oxidation of cycloalkanes is obtained when the molar ratio of gold to palladium in the alloy is in the range from about 1:18 to about 18:1, more preferably from about 1:15 to about 15:1, more preferably from about 1:12 to about 12:1, more preferably from about 1:10 to about 10:1, and most preferably from about 1:9 to about 9:1.

The support has a very significant effect upon the catalytic performance of the gold-palladium particles in the oxidation of cycloalkanes. For example, a silica-supported AuPd catalyst was found to be inactive in the cyclohexane oxidation reaction, whereas replacement of the silica with silicon nitride (Si₃N₄) as the support provided good catalytic activity for the AuPd catalyst in cyclohexane oxidation. Thus, the interaction between support, particularly surface oxygen species, and active species is a key factor for catalytic activity. Surprisingly, the inventors observed that, using some supports, additional amounts of catalysts did not improve the catalytic performance, but quenched the whole reaction. This phenomenon would appear contradictory to conventional catalyst performance since a greater amount of catalyst would normally be expected to improve the concentration of active oxygen species, facilitating the oxidation of the reactant. The phenomenon is not, however, unknown. The so-called catalyst-inhibitor transition was identified by J. F. Black (JACS, 1978, 100, 527-535) who observed sharp changes in catalytic performance dependent on the concentration of a cobalt/manganese salt in a metal-catalysed autoxidation.

The catalyst-inhibitor transition is of particular importance in an industrial process. Catalyst systems associated with a catalyst-inhibitor transition which is very sensitive to catalyst amount requires the presence of precise amounts of catalysts for industrial production. Such catalyst systems are inflexible and prone to negative effects on productivity, and hence unattractive for an industrial process.

The catalyst system of the invention utilises a support selected from the group consisting of carbides, nitrides and certain oxides. Preferably, the carbide supports are selected from B₄C₃, Mo₂C, ZrC, WC, SiC and TiC, more preferably from B₄C₃ and SiC, and more preferably the carbide is SiC. Preferably, the nitrides are selected from BN, C₃N₄, AlN and Si₃N₄, and preferably from BN and Si₃N₄. The oxides of magnesium, zinc and aluminium may be single metal oxides or mixed metal oxides, for instance selected from MgO, Al₂O₃, ZnO, and MgAl₂O₄. Preferably, the catalyst system of the invention utilises a carbide or nitride support, more preferably a carbide support. The carbide supports exhibit high stability and chemical inactivity in cycloalkane oxidation, even at higher temperature, and were found to be superior in these respects to, for instance, the nitride supports. Thus, the carbide support is effectively inert for cycloalkane oxidation, and is neither catalyst nor inhibitor. A particular advantage of the present invention is that the preferred supports (most notably the carbide supports) modify catalyst performance in a way which greatly improves the resistance of the catalyst system to catalyst amount, and are therefore particularly suitable for an industrial process.

The loading of the AuPd catalyst is preferably in the range of from about 0.1 to about 10 wt % based on the total weight of the support and catalyst, preferably from about 0.5 to about 5 wt %, preferably from about 0.5 to about 2 wt. %.

The inventors also found that modification of the AuPd catalyst by doping further improves catalytic performance. Such a modification combines the improved resistance of the catalyst system to catalyst amount with increased conversion and/or selectivity. Furthermore, the doping modification inhibits the production of ring-opened carboxylic acid species, such as adipic acid in the oxidation of cyclohexane. This is advantageous because adipic acid and other by-products would otherwise have to be separated from the reaction mixture.

The introduction of the dopant is typically effected by surface modification of the supported AuPd catalyst, using methods as described herein, for instance by coating the surface of the supported AuPd catalyst. Preferred dopants are the basic metal oxides and hydroxides, and in particular MgO, Al₂O₃, ZnO, CaO, Mg(OH)₂, Al(OH)₃, Zn(OH)₂, and Ca(OH)₂. Particularly effective dopants are selected from Mg(OH)₂ and Al(OH)₃, preferably Al(OH)₃. As used herein, the term “dopant” refers to a material which is different to the materials of the support and the catalyst. Thus, a basic metal oxide or hydroxide in the list hereinabove is used as a “dopant” in a supported catalyst which does not contain that species as the support. The basic metal oxides and hydroxides in the list hereinabove are of particular utility in supported AuPd catalysts wherein the support is selected from the carbides and nitrides described herein.

The amount of dopant introduced into the supported AuPd catalyst is preferably quantified as a function of the amount of dopant used during the catalyst preparation, and defined as the weight percent of dopant relative to the total weight of the initial aqueous solution of gold and palladium salts used in the preparation. Thus, the amount of dopant used during the catalyst preparation is preferably in the range of from about 1.25×10⁻³wt % to about 2×10⁻² wt %, preferably from about 1.5×10⁻³wt % to about 1×10⁻² wt %, and in one embodiment about 5×10⁻³ wt %, relative to the total weight of the initial aqueous solution of gold and palladium salts used in the preparation.

The supported catalyst is suitable for use as a heterogeneous catalyst in the process for oxidising cycloalkane. The heterogeneous nature of the catalyst is advantageous, allowing the catalyst to be more easily separated from the reaction products and recycled. For example, when the cycloalkane is cyclohexane, the reactant is in the liquid phase and the catalyst is in the solid thereby allowing the catalyst to be extracted from the reaction mixture by filtration.

Thus, in a fourth aspect, the present invention provides the use of the supported catalyst defined herein as a catalyst, suitably a heterogeneous catalyst, in a process for oxidising cycloalkane(s), particularly for preparing cycloalkanol(s) and/or cycloalkanone(s).

Preparation of the Supported AuPd Catalyst

The gold-palladium particles of the catalyst are preferably prepared according to the sol-immobilisation method defined hereinafter, in which a water-soluble polymer is added to a solution of gold and palladium salts, with the subsequent addition of a reducing agent. The resulting gold-palladium particles are obtained as a sol and then supported on a support. The supports are usually added to the sol, as a solid, under vigorous stirring conditions for up to about 3 hours. The supported catalyst can be extracted by filtration, washed with water, and then dried. Typical drying temperatures are around 120° C., and drying may be conducted for 8-12 hours. Accordingly, the sol-immobilisation method for preparing the catalyst of the invention preferably comprises the following steps:
(a) preparing an aqueous solution of gold and palladium salts;
(b) adding a water-soluble polymer to the solution obtained in step (a);
(c) adding a reducing agent to the solution obtained in step (b) to form a sol; and
(d) adding a support to the sol solution obtained in step (c) to form a slurry.

The process for preparing the supported catalyst of the invention may further comprise, additionally to steps (a) to (d) above, the following steps:
(e) filtering the resulting slurry obtained in step (d);
(f) washing the product of step (e) with water; and
(e) drying the washed product of step (f).

Any suitable palladium salt may be used, and preferably the palladium salt is a palladium (II) salt, for instance PdCl₂, which is commonly used for the production of palladium catalysts. Any suitable gold salt may be used, and preferably the gold salt is a gold (III) salt, for instance gold (III) chloride, KAuCl₄ or chloroauric acid (HAuCl₄), used herein in the form of its trihydrate HAuCl₄.3H₂O.

Any suitable reducing agent may be used, and in a preferred embodiment the reducing agent is NaBH₄. The reducing agent is preferably provided in a molar excess with respect to the amount of gold, and in one embodiment in a molar ratio of at least about 2:1, and in a further embodiment in a molar ratio of 5:1. The reducing agent is normally provided in the form of an aqueous solution, and in one embodiment it is provided as a 0.1 M aqueous solution.

The water-soluble polymer is preferably polyvinylalcohol (PVA). The PVA may be provided in partially or fully hydrolysed form, and in one embodiment is partially hydrolysed. The PVA suitably exhibits a degree of hydrolysis of at least about 70%, and in one embodiment a degree of hydrolysis in the range of from about 70 to about 90%. In a preferred embodiment, the water-soluble polymer has a molecular weight of from about 5000 to about 20,000, and preferably from about 8,000 to about 12,000. The water-soluble polymer is normally provided in the form of an aqueous solution, and in one embodiment it is provided as a 1 wt % aqueous solution. The water-soluble polymer is preferably provided in weight excess relative to the amount of gold, and in one embodiment it is provided in a weight ratio of 1.2:1 with respect to the amount of gold. The ratio of PVA to the metallic species of the catalyst (AuPd) is preferably from about 0.01:1 to about 0.1:1.

The doped supported gold-palladium catalysts of the invention are preferably prepared using the sol-immobilisation method described herein process, comprising the additional step of introducing into the process a solution (normally aqueous) of a salt comprising the metal ion of the dopant, typically after production of the sol (step (c)) and before addition of the support (step (d)). Metal nitrate salts are particularly suitable, and magnesium nitrate and aluminium nitrate were used herein to generate the magnesium hydroxide and aluminium hydroxide dopants, respectively. Subsequently, the support is added as described herein and the pH value of the solution adjusting to a value between 8 and 12 using any suitable base, for instance ammonia. After vigorous stirring, the supported catalyst is filtered, washed and dried as described above.

In one embodiment, the supported catalysts may be calcined after drying. Calcination can be conducted under an atmosphere of air, nitrogen, hydrogen, helium or the like, and is typically conducted under an atmosphere of air. Calcination temperatures may range from 200 to 1000° C., typically 200 to 700° C. The calcination time may be from about 1 to about 40 hours, more typically from about 2 to about 15 hours. In a preferred embodiment, however, the supported catalysts (particularly the doped supported catalysts) of the present invention are not calcined, since while selectivity may increase, conversion tends to decrease.

Alternative methods for preparation of the supported catalyst include impregnation methods, precipitation methods and seed-mediated growth, and these methods may use the reactants described hereinabove.

In an impregnation method, an aqueous solution of gold and palladium salts is prepared, and the support added thereto in the desired weight ratios. The suspension is then stirred, filtered and washed. The supported catalyst is then dried, as described above for the sol-immobilisation method, and consecutively calcined, as described above. The aerobic calcination process reduces the impregnated gold and palladium salt precursors to provide gold-palladium particles.

In a precipitation method, an aqueous solution of gold and palladium salts is prepared, to which is added a suitable base (for instance, sodium carbonate) with stirring until a pH of from about 9 to about and 11 is attained. The support is added with continuous stirring for up to about 3 hours, maintaining the pH between 9 and 11. The mixture is heated from room temperature to around 70° C., and a suitable reducing agent (for instance formaldehyde) is then added. The solid is filtered, washed and dried, as described above.

Oxidation of Cycloalkanes

The supported catalysts described herein are particularly suitable for oxidising cycloalkanes to the corresponding cycloalkanol and/or cycloalkanone.

Examples of the cycloalkane as the raw material include monocyclic cycloalkanes having no substituent on the ring, such as cyclopropane, cyclobutane, cyclopentane, cyclohexane, cycloheptane, cyclooctane, cyclodecane, cyclododecane and cyclooctadecane; polycyclic cycloalkanes such as decalin and adamantane; and cycloalkanes having a substituent on the ring, such as methylcyclopentane and methylcyclohexane. Mixtures of cycloalkanes may be used, although it is preferable to limit the reaction to a single substrate, wherever possible, in order to avoid cross-reactions and to facilitate isolation of the target compounds. The present invention is of particular commercial utility for the oxidation of cyclohexane or cyclododecane, particularly cyclohexane.

An oxygen-containing gas is usually used as the oxygen source. This oxygen-containing gas may be, for example, air, pure oxygen, or air or pure oxygen diluted with an inert gas such as nitrogen, argon or helium. Oxygen-enriched air may also be used.

The amount of the supported catalyst to be used is usually in the range from about 0.01 to about 50 parts by weight, and preferably from about 0.1 to about 10 parts by weight, based on 100 parts by weight of cycloalkane.

The reaction temperature is usually no more than about 200° C., preferably no more than 180° C., and typically from about 50° C. to about 150° C., and preferably form about 100° C. to about 150° C.

The reaction pressure is usually from about 0.01 to about 10 MPa, and preferably from about 0.1 to about 2 MPa. The duration of the reaction is typically no more than 24 hours, and typically in the range of from about 1 to about 20 hours, preferably 1 to 5 hours. A solvent may be used for the reaction, and suitable solvents include nitrile solvents such as acetonitrile and benzonitrile, and carboxylic acid solvents such as acetic acid and propionic acid. In a preferred embodiment, the reaction is carried out in the absence of solvent.

The oxidation reaction in the presence of the supported AuPd catalyst can also be operated in the presence of a radical initiator. Examples of the radical initiator include azonitrile compounds such as 2,2′-azobis(isobutyronitrile), 2,2′-azobis(2,4-dimethylvarelonitrile), and 2,2′-azobis(4-methoxy-2,4-dimethylvarelonitrile); and peroxides such as TBHP, peroxydibenzoyl, peroxydilauroyl, t-butylperoxy 2-ethylhexanoate, and bis(2-ethylhexyl)peroxydicarbonate. Other examples of initiators include cyclohexanone, N-hydroxyphthalimide, and 2-butanone. Two or more kinds of these radical initiators may be used in combination, or a single radical initiator may be used. When the radical initiator is used, the amount is usually from 0.1 mole or less per mole of cycloalkane. However, a commercial process (particularly a continuous process) preferably does not use such a radical initiator, the exception being the use of an initiator which corresponds to the target cycloalkanone. Thus, in one embodiment, the oxidation process of the present invention is operated in the presence of an initiator which is the target cycloalkanone of the oxidation of the cycloalkane feedstock; for example, where the feedstock is cyclohexane, then cyclohexanone may be added into the reaction mixture as a radical initiator. The addition of the target cycloalkanone as initiator is typically effected by recycle of a portion of the target cycloalkanone product back to the reactor.

Once the oxidation reaction is completed, conventional post-treatment steps may be conducted. Thus, the reaction mixture is typically filtered to separate the catalyst, followed by washing with water and further distillation.

In a continuous commercial process, the oxidation reaction may be carried out using conventional oxidation reactors known in the art. For example, those described in U.S. Pat. Nos. 3,957,876; 3,510,526 and 3,530,185.

The invention is further illustrated by the following examples. The examples are not intended to limit the invention as described above. Modification of detail may be made without departing from the scope of the invention.

### EXAMPLES

The following general methods were used to synthesise the catalysts listed in Table 1.

Preparation of Supported Au/Pd Catalysts (Sol-Immobilization Method)

An aqueous solution of PdCl₂ (Johnson Matthey) and/or HAuCl₄.3H₂O of the desired concentration was prepared. Polyvinylalcohol (PVA) (1 wt % solution; Aldrich; MW=10 000; 80% hydrolyzed) was added (PVA/Au (by wt)=1.2), and a 0.1 M freshly prepared solution of NaBH₄ (Aldrich, NaBH₄/Au (mol/mol)=5) was then added to form a dark-brown sol. 30 minutes after sol generation, the colloid was immobilized by adding the desired amount of support under vigorous stirring conditions. After 2 h the slurry was filtered, the catalyst washed thoroughly with distilled water (neutral mother liquors) and dried at 120° C. overnight.

Surface Modification of Supported Au/Pd Catalysts by Doping with Mg(OH)₂ or Al(OH)₃

The modified catalysts were prepared by using a modified sol-immobilization method. After formation of the Au—Pd nanoparticles in the sol, the desired amount (herein 10, 40, 80 or 160 mg) of magnesium nitrate or aluminium nitrate was added into the solution. Afterwards, the desired amount of support was added and the pH value of the solution was adjusted to 11 by adding ammonia. After two hours stirring, the filtrate was washed and dried at 120° C. overnight.

TABLE 1

| | | | | | Surface | |
|---|---|---|---|---|---|---|
| | | | Molar | | modification | |
| Cat. | | | ratio of | | (mg) | |
| No. | Catalyst label | Weight (%) | Au/Pd | Supports | Mg(OH)₂ | Al(OH)₃ |
| 1 | 1% AuPd/MgO | 1 | 1 | MgO | | |
| 2 | 1% AuPd/Al₂O₃ | 1 | 1 | Al₂O₃ | | |
| 3 | 1% AuPd/TiO2 | 1 | 1 | TiO₂ | | |
| 4 | 1% AuPd/Zeolite | 1 | 1 | Zeolite | | |
| 5 | 1% AuPd/SiO₂ | 1 | 1 | SiO₂ | | |
| 6 | 1% AuPd/AC | 1 | 1 | Activated | | |
| | | | | carbon | | |
| 7 | 1% AuPd/DVB | | | DVB | | |
| 8 | 1% AuPt/MgO | 1 | 1 | MgO | | |
| 9 | 1% AuAg/MgO | 1 | 1 | MgO | | |
| 10 | 1% Au₂Pd₁/MgO | 1 | 2 | MgO | | |
| 11 | 1% Au₁Pd₂/MgO | 1 | 0.5 | MgO | | |
| 12 | 1% Au₄Pd₁/MgO | 1 | 4 | MgO | | |
| 13 | 1% Au₁Pd₄/MgO | 1 | 0.25 | MgO | | |
| 14 | 1% Au₉Pd₁/MgO | 1 | 9 | MgO | | |
| 15 | 1% Au₁Pd₉/MgO | 1 | 1/9 | MgO | | |
| 16 | 1% Au₂₀Pd₁/MgO | 1 | 20 | MgO | | |
| 17 | 1% Au₁Pd₂₀/MgO | 1 | 1/20 | MgO | | |
| 18 | 1% AuPd/ZnO | 1 | 1 | ZnO | | |
| 19 | 1% AuPd/MgAl₂O₄ | 1 | 1 | MgAl₂O₄ | | |
| 20 | 1% AuPd/Si₃N₄ | 1 | 1 | Si₃N₄ | | |
| 21 | 1% AuPd/BN | 1 | 1 | BN | | |
| 22 | 1% AuPd/SiC | 1 | 1 | SiC | | |
| 23 | 1% AuPd/B₄C₃ | 1 | 1 | B₄C₃ | | |
| 24 | 1% AuPd/M40-SiC | 1 | 1 | SiC | 40 | |
| 25 | 1% AuPd/A10-SiC | 1 | 1 | SiC | | 10 |
| 26 | 1% AuPd/A40-SiC | 1 | 1 | SiC | | 40 |
| 27 | 1% AuPd/A80-SiC | 1 | 1 | SiC | | 80 |
| 28 | 1% AuPd/A160-SiC | 1 | 1 | SiC | | 160 |
| 29 | 1% Au₄Pd₁/A40-SiC | 1 | 1 | SiC | | 40 |
| 30 | 1% Au₄Pd₁/M40-SiC | 1 | 4:1 | SiC | 40 | |
| 31 | 1% Au₉Pd₁/A40-SiC | 1 | 9:1 | SiC | | 40 |
| 32 | 1% Au₁Pd₉/A40-SiC | 1 | 1:9 | SiC | | 40 |

Oxidation of Cyclohexane

The catalytic activity of the prepared catalysts in the oxidation of cyclohexane oxidation was studied on a laboratory scale by the method described below. Catalytic oxidation tests were performed using a glass bench reactor, which was connected to a cylinder of O₂ gas. After the addition of cyclohexane (10 mL) and desired amount of catalyst had been added to the unit, reactants were magnetically stirred at 140° C. and 3 bar O₂ for 17 hours. After reaction was complete, the desired amount of chlorobenzene was added into the product as an external standard. The liquid products were then injected into a Gas Chromatograph (Varian 3200) with a CP-Wax 42 column and FID detector for ketone, alcohol, peroxide, ether and ester quantification. Any solid products of the reaction present in the final mixture were collected by filtration, washed with cyclohexane and subsequently dissolved in a known weight of methanol. Subsequently, 300 μL of sample out of the 10 mL product solution was mixed with 2 ml of 14% boron tri-fluoride (BF₃) in methanol, which was subsequently heated at 70° C. and magnetically stirred for half hour. After complete conversion of the acid products to corresponding methyl esters, the reaction was stopped by adding 2 mL water. Finally, the esters formed were extracted from the mixture using a known volume of dichloromethane and injected into GC for quantification. The results are described below.

I: Effects of Different Supports on the Activity of AuPd Catalyst

The catalytic performance in the oxidation of cyclohexane of Au-based catalysts on different supports was tested in accordance with the procedure above, using 6 mg of the catalyst. The results presented in Table 2 below demonstrate that the AuPd nano-alloys can display significant catalytic activity, but that this is highly dependent on the support. The best performance of 11% conversion and 97% selectivity was obtained by using MgO supported

Au Pd, which is even better than that of the commercial catalyst, cobalt naphthenate. Some of the supports, for instance, silica or zeolite, quenched the whole reaction. In this series of experiments, the performance of the MgO-supported AuPd catalyst was compared with AuAg and AuPt nano-alloys, which displayed inferior catalytic performance.

TABLE 2

| Expt. | Catalyst | Conversion | Selectivity (%) | | | | |
|---|---|---|---|---|---|---|---|
| No. | Identity | (%) | Ket. | Alc. | CHHP | AA | Total |
| 1 | 1% AuPd/ | 11 | 40 | 55 | 0 | 2 | 97 |
| | MgO | | | | | | |
| 2 | 1% AuPd/ | 6 | 40 | 41 | 1 | 7 | 90 |
| | Al₂O₃ | | | | | | |
| 3 | 1% AuPd/ | 4.5 | 28 | 35 | 0 | 9 | 72 |
| | TiO₂ | | | | | | |
| 4 | 1% AuPd/ | 0.7 | 22 | 21 | 0 | 0 | 43 |
| | Zeolite | | | | | | |
| 5 | 1% AuPd/ | 0 | — | — | — | — | — |
| | SiO₂ | | | | | | |
| 6 | 1% AuPd/ | 1 | 20 | 61 | 0 | 1 | 82 |
| | AC | | | | | | |
| 7 | 1% AuPd/ | 0 | — | — | — | — | — |
| | DVB | | | | | | |
| 8 | 1% AuPt/ | 1.6 | 11 | 23 | 6 | 0 | 41 |
| | MgO | | | | | | |
| 9 | 1% AuAg/ | 5.7 | 26 | 44 | 6 | 15 | 90 |
| | MgO | | | | | | |
| 10 | Control | 1.1 | 19 | 22 | 57 | | 98 |
| | (no catalyst) | | | | | | |
| 11 | Cobalt | 11 | 37 | 45 | 1 | 2 | 85 |
| | napthenate | | | | | | |

Ket. = cyclohexanone;
Alc. = cyclohexanol;
CHHP = cyclohexyl hydrogen peroxide;
AA = adipic acid

II: Effect of Au:Pd Ratio

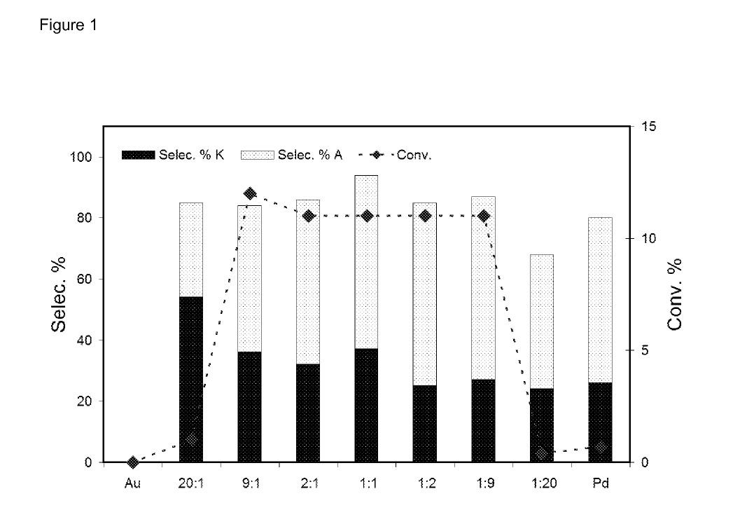

A series of experiments was conducted to determine the optimum molar ratios of gold and palladium in the catalyst, using MgO as the support. The results are shown in FIG. 1, and demonstrate that the preferred Au:Pd molar ratio is in the range from about 1:9 to about 9:1.

III: Effect of Catalyst Amount

In experiments designed to optimise the reaction conditions with the MgO supported catalyst (1% AuPd/MgO), it was observed that increasing the catalyst amount from 0 to 6 mg resulted in high selectivity with increasing conversion levels. Surprisingly, however, further amount of catalyst did not improve the catalytic performance but instead quenched the whole reaction.

Thus, when Experiment No. 1 in Table 2 was re-run using 8mg of catalyst, no cyclohexane oxidation was observed (0% conversion).

IV: Effects of Different Supports on the Resistance of AuPd Catalysts to Catalyst Dose

The influence of the support on the resistance of the AuPd catalyst to catalyst amount was investigated using oxide, nitride and carbide supports. The oxidation reactions were run in accordance with the procedure describe above, with variable amounts of the supported catalyst. The results, which are presented in Table 3 below, demonstrate that the carbide-supported catalysts display superior resistance to high dose of catalyst.

TABLE 3

| Expt | | Amt. | Conversion | Selectivity (%) | | | | |
|---|---|---|---|---|---|---|---|---|
| No. | Catalyst | (mg) | (%) | Ket. | Alc. | CHHP | AA | total |
| 12a | 1% AuPd/ZnO | 6 | 7.8 | 44 | 36 | | 5 | 86 |
| 12b | | 10 | 9 | 41 | 32 | | 7 | 82 |
| 12c | | 20 | 5.4 | 42 | 30 | | 14 | 86 |
| 12d | | 30 | 0 | — | — | | — | — |
| 13a | 1% AuPd/Al₂O₃ | 6 | 7 | 43 | 33 | | 6 | 83 |
| 13b | | 10 | 11 | 31 | 44 | | 6 | 82 |
| 13c | | 20 | 0 | — | — | | — | — |
| 14a | 1% AuPd/MgAl₂O₄ | 3 | 8.3 | 28 | 41 | | 16 | 85 |
| 14b | | 6 | 6 | 46 | 35 | | 5 | 88 |
| 14c | | 10 | 6 | 36 | 36 | | 6 | 80 |
| 14d | | 20 | 0 | — | — | | — | — |
| 15a | 1% AuPd/Si₃N₄ | 6 | 9 | 37 | 31 | | 12 | 82 |
| 15b | | 10 | 6.5 | 37 | 31 | | 22 | 91 |
| 15c | | 20 | 6.5 | 35 | 29 | | 24 | 89 |
| 15d | | 30 | 5 | 34 | 41 | | 18 | 19 |
| 15e | | 40 | 0 | — | — | | — | — |
| 16a | 1% AuPd/BN | 6 | 8 | 38 | 38 | | 11 | 89 |
| 16b | | 10 | 7.5 | 34 | 36 | | 10 | 80 |
| 16c | | 20 | 3.2 | 37 | 62 | | 0 | 98 |
| 16d | | 30 | 0 | — | — | | — | — |
| 17a | 1% AuPd/SiC | 3 | 8.8 | 41 | 36 | | 8 | 87 |
| 17b | | 6 | 6.7 | 45 | 32 | | 10 | 90 |
| 17c | | 10 | 8 | 38 | 34 | | 8 | 83 |
| 17d | | 20 | 7.5 | 36 | 43 | | 18 | 98 |
| 17e | | 30 | 8 | 32 | 35 | | 17 | 85 |
| 17f | | 40 | 6.4 | 30 | 34 | | 19 | 83 |
| 17g | | 50 | 6 | 35 | 38 | | 16 | 89 |
| 17h | | 60 | 6 | 37 | 30 | | 11 | 70 |
| 17i | | 80 | 5.4 | 33 | 33 | | 12 | 80 |
| 18 | SiC | 6 | 1.6 | 31 | 19 | | 14 | 64 |
| 19a | 1% AuPd/B₄C₃ | 6 | 7 | 44 | 44 | | 4 | 93 |
| 19b | | 10 | 6 | 47 | 39 | | 5 | 92 |
| 19c | | 20 | 3 | 33 | 39 | | 11 | 86 |
| 19d | | 40 | 3 | 41 | 46 | | 4 | 91 |

V: Catalytic Performance of Carbide-Supported AuPd Dosed with Mg(OH) and Al(OH)₃

The catalytic performance of the carbide-supported AuPd catalysts was improved by the introduction of magnesium or aluminium oxides into the supported catalysts. The results of cyclohexane oxidation performed as described herein, using varying amounts of catalyst, are presented in Table 4.

TABLE 4

| Expt | | Amount | Conversion | Selectivity % | | | | |
|---|---|---|---|---|---|---|---|---|
| No. | Catalyst | (mg) | (%) | Ket. | Alc. | CHHP | AA | total |
| 20a | 1% AuPd/M40-SiC | 6 | 5.8 | 51 | 30 | | 11 | 92 |
| 20b | | 10 | 9 | 38 | 43 | | 7 | 90 |
| 20c | | 20 | 9 | 38 | 52 | | 4 | 94 |
| 20d | | 40 | 7 | 40 | 49 | | 4 | 94 |
| 21a | 1% AuPd/A10-SiC | 6 | 8 | 32 | 39 | | 13 | 84 |
| 21b | | 10 | 8 | 29 | 39 | | 13 | 82 |
| 21c | | 20 | 5.2 | 23 | 48 | | 19 | 90 |
| 21d | | 40 | 5 | 22 | 43 | | 20 | 86 |
| 22a | 1% AuPd/A40-SiC | 6 | 12 | 28 | 54 | | 4 | 88 |
| 22b | | 10 | 10 | 34 | 51 | | 4 | 90 |
| 22c | | 20 | 8 | 29 | 54 | | 7 | 91 |
| 22d | | 40 | 3 | 44 | 50 | | 0 | 95 |
| 23a | 1% AuPd/A80-SiC | 6 | 9 | 41 | 44 | | 7 | 93 |
| 23b | | 10 | 8 | 43 | 45 | | 4 | 92 |
| 23c | | 20 | 7 | 49 | 41 | | 4 | 95 |
| 23d | | 40 | 6 | 38 | 58 | | | 96 |
| 24a | 1% AuPd/A160-SiC | 6 | 8 | 47 | 28 | | 9 | 84 |
| 24b | | 10 | 6 | 52 | 46 | | | 98 |
| 24c | | 20 | 5 | 40 | 52 | | | 91 |
| 24d | | 40 | 5 | 50 | 45 | | | 94 |

The designations M40, A10, A40, A80 and A160 refer to the identity and amount of base dopant used (A=Al(OH)₃; M=Mg(OH)₂; and the number is the total weight of base dopant present during the preparation method.

VI: Effect of Au:Pd Ratio of Carbide-Supported AuPd Catalysts Dosed with Mg(OH)₂ and Al(OH)₃

Further oxidation reactions were run to determine the effect of the Au:Pd ratio on the doped carbide-supported AuPd catalysts. The results are presented in Table 5 below.

TABLE 5

| Expt | | Amount | Conversion | Selectivity % | | | | |
|---|---|---|---|---|---|---|---|---|
| No. | Catalysts | (mg) | (%) | Ket. | Alc. | CHHP | AA | total |
| 25a | 1% Au₄Pd₁/M40-SiC | 6 | 7 | 60 | 34 | | 4 | 98 |
| 25b | | 10 | 7 | 46 | 30 | | 9 | 87 |
| 25c | | 20 | 7 | 38 | 52 | | 4 | 94 |
| 25d | | 40 | 8.7 | 38 | 43 | | 4 | 86 |
| 26a | 1% Au₄Pd₁/A40-SiC | 6 | 8.5 | 40 | 40 | | 8 | 88 |
| 26b | | 10 | 9 | 39 | 39 | | 4 | 83 |
| 26c | | 20 | 8 | 41 | 42 | | 4 | 88 |
| 26d | | 40 | 5.7 | 49 | 45 | | 5 | 99 |
| 27a | 1% Au₉Pd₁/A40-SiC | 6 | 9 | 41 | 46 | | 0 | 88 |
| 27b | | 10 | 10 | 38 | 47 | | 0 | 87 |
| 27c | | 20 | 11 | 37 | 57 | | 0 | 96 |
| 27d | | 40 | 10 | 35 | 49 | | | 84 |
| 28a | 1% Au₁Pd₉/A40-SiC | 6 | 7 | 42 | 38 | | 4 | 84 |
| 28b | | 10 | 8 | 42 | 42 | | 4 | 88 |
| 28c | | 20 | 11 | 51 | 38 | | 2 | 91 |
| 28d | | 40 | 7 | 45 | 42 | | | 87 |

1. An oxidation process for preparing cycloalkanol(s) and/or cycloalkanone(s) comprising contacting one or more cycloalkane(s) with an oxidant in the presence of a supported catalyst wherein the oxidant is an oxygen-containing gas and the supported catalyst comprises a catalyst comprising gold-palladium particles and a support selected from carbides, nitrides and oxides, wherein the oxides are selected from magnesium, aluminium and zinc oxides.

2. A process for oxidising cycloalkanes comprising contacting one or more cycloalkane(s) with an oxidant in the presence of the supported catalyst wherein the oxidant is an oxygen-containing gas and the supported catalyst comprises a catalyst comprising gold-palladium particles and a support selected from carbides, nitrides and oxides, wherein the oxides are selected from magnesium, aluminium and zinc oxides.

3. (canceled)

4. (canceled)

5. The process according to claim 1, wherein the gold-palladium particles are nanoparticles exhibiting a mean longest diameter of no more than 200 nm.

6. The process according to claim 1, wherein the molar ratio of gold to palladium is in the range from about 1:15 to about 15:1.

7. The process according to claim 1, wherein the support is selected from carbides and nitrides.

8. The process according to claim 1, wherein the support is a carbide support selected from B₄C₃, Mo₂C, ZrC, WC, SiC and TiC.

9. The process according to claim 1, wherein the support is a nitride support selected from BN, C₃N₄, AlN and Si₃N₄.

10. The process according to claim 1, wherein the support is an oxide support selected from MgO, Al₂O₃, ZnO, and MgAl₂O₄.

11. The process according to claim 1, wherein the loading of the AuPd catalyst is in the range of from about 0.1 to about 10 wt % based on the total weight of the support and catalyst.

12. The process according to claim 1, wherein the supported AuPd catalyst further comprises a dopant selected from basic metal oxides and hydroxides.

13. The process according to claim 1, wherein the cycloalkane is cyclohexane or cyclododecane.

14. The process according to claim 1, wherein the amount of supported catalyst is in the range from about 0.01 to about 10 parts by weight, based on 100 parts by weight of cycloalkane.

15. The process according to claim 1, wherein the reaction temperature is no more than 200° C., and/or the reaction pressure is from 0.01 to 10 MPa, and/or the duration of the reaction is in the range of from 1 to 20 hours.

16. The process according to claim 1, wherein the oxidation process is operated in the presence of an initiator which is the target cycloalkanone of the oxidation reaction.

17. A process according to claim 2, wherein the gold-palladium particles are nanoparticles exhibiting a mean longest diameter of no more than 200 nm

18. A process according to claim 2, wherein the molar ratio of gold to palladium is in the range from about 1:15 to about 15:1.

19. A process according to claim 2, wherein the support is selected from carbides and nitrides.

20. A process according to claim 2, wherein the support is a carbide support selected from B₄C₃, Mo₂C, ZrC, WC, SiC and TiC.

21. A process according to claim 2, wherein the support is a nitride support selected from BN, C₃N₄, AlN and Si₃N₄.

22. A process according to claim 2, wherein the support is an oxide support selected from MgO, Al₂O₃, ZnO, and MgAl₂O₄.

23. A process according to claim 2, wherein the loading of the AuPd catalyst is in the range of from about 0.1 to about 10 wt % based on the total weight of the support and catalyst.

24. A process according to claim 2, wherein the sAuPd catalyst further comprises a dopant selected from basic metal oxides and hydroxides

25. A process according to claim 2, wherein the cycloalkane is cyclohexane or cyclododecane.

26. A process according to claim 2, wherein the amount of supported catalyst is in the range from about 0.01 to about 10 parts by weight, based on 100 parts by weight of cycloalkane.

27. A process according to claim 2, wherein the reaction temperature is no more than 200° C., and/or the reaction pressure is from 0.01 to 10 MPa, and/or the duration of the reaction is in the range of from 1 to 20 hours.

28. A process according to claim 2, wherein the oxidation process is operated in the presence of an initiator which is the target cycloalkanone of the oxidation reaction.
